(19) United States
(12) Patent Application Publication
(10) Pub. No.: US 2003/0109699 A1
(43) Pub. Date: Jun. 12, 2003

(54) Amphiphilic quinolylpolyamines as transfer agents for biologically active macromolecules

(76) Inventor: Oliver Keil, Schildow (DE)

Correspondence Address: BRUCE LONDA, NORRIS, MCLAUGHLIN & MARCUS, P.A., 220 EAST 42ND STREET, 30TH FLOOR, NEW YORK, NY, 10017, US

(21) Appl. No.: 10/149,732

(22) PCT Filed: Dec. 13, 2000
(86) PCT No.: PCT/DE00/04443

(30) Foreign Application Priority Data
Dec. 17, 1999 (DE) 19960924.1

(51) Int. Cl. C07J 43/00

(52) U.S. Cl. 540/107

(57) ABSTRACT

The present invention describes amphiphilic polyamines and salts thereof, which are able to form aggregates or particles with biopolymers like DNA, RNA, antisense oligonucleotides, ribozymes, proteins and peptides capable of transporting the bio-polymers into eucaryotic cells. In this regard polyaminoquinoline derivatives, which have been modified with lipophilic groups, have been proven particularly useful. Due to their property to form aggregates with biologically active molecules like DNA or RNA, these compounds are especially suitable for gene therapy applications but also for diagnostic purposes.

The present invention entails amphiphilic polyamines and salts thereof, that can facilitate the transport of biologically active macromolecules (in particular DNA and RNA) into eucaryontic cells.

Over the last 10 years the transfer of biopolymers, in particular of DNA, RNA and oligonucleotides into eucaryotic cells has developed into a distinct field in molecular biology and molecular medicine (P. A. Martin, S. M. Thomas; Human Gene Therapy 9, 87-114 (1998). Of particular interest are strategies for gene therapy as well as diagnostic methods. Established procedures to introduce DNA into eucaryotic cells relay on the incorporation of the particular DNA fragment into replication deficient recombinant retro-, adeno- or adeno-associated viruses. The disadvantage of this approach is the restriction to almost exclusively ex vivo application, since viral proteins are resulting in major immune responses. Furthermore, mutations leading to replication competent viruses cannot be excluded entirely. An additional disadvantage of retro-viruses is the unspecific but stable integration into the host genome that can lead to potentially malignant mutations. Based on these properties it is understandable that the viral vector systems demand extremely high safety requirements that can only be fulfilled with high cost. Biophysical methods such as the Gene gun using gold particles loaded with DNA (“Biolistik”) or electroporation can obviously only be employed ex-vivo. The well-known Calcium phosphate co-precipitation and the DEAE-dextran method are not suitable because of their low gene transfer efficiency.

Another method developed over the last few years is using cationic polymers such as poly-L-lysine, polyethylenimine or PAMAM-dendrimers, to transport biologically active macromolecules into eucaryotic cells. Poly-anions such as DNA, RNA or oligonucleotides are forming aggregates with the poly-cations through electrostatic interactions. Such aggregates are entering the cells probably via endocytosis.

However, in the case of poly-L-lysine introduction of receptor ligands (e.g. transferrin, glycoprotein) (E. Wagner et al., Proc. Natl. Adad. Sci. USA 87, 3410-3414 (1990) or peptides causing lysis of the endosomes (E. Wagner, Proc. Natl. Acad. Sci. USA 89, 7934-7938 (1992) are necessary to obtain sufficient transfection efficiency. These receptor ligands have to be attached to poly-L-lysine through chemical reactions. The necessity of chemical modification and the polymeric character of the compounds result in heterogeneous mixtures and therefore their synthesis in a more defined form is very labour-intensive.

Another transfer system that gained importance over the past few years is based on the work published by Felgner et al. Here, cationic lipidic amphiphiles either pure or in mixtures with neutral phospholipids such as dioleoylphosphatidylethanolamine (DOPE) form liposomal or micellar structures. Together with anionic biopolymers (such as DNA or RNA) these structures form aggregates through electrostatic interactions, that are taken up efficiently by eucaryotic cells. In this way the DNA can be transported into the nucleus leading to expression of the corresponding protein. The exact mechanism is so far unknown, however, there is general agreement that the aggregates are reaching the interior of the cells via the endosomes and endocytotic processes. Furthermore, it is known that the pH in the endosomes drops to approximately pH 5-6 over time as a result of H⁺-ATPases in vacuoles. There are indications that the increase in proton concentration is the cause for the subsequent fusion of endosomes with lysosomes (A. K. Fok et al., Eur. J. Cell Biol. 43 (3), 412-420 (1987)). For the internalized biopolymers to reach other cell compartments (e.g. cytoplasm or cell nucleus) it is necessary that the biopolymers escape the endosomes or otherwise they will be digested enzymatically in the lysosomes. The escape from the endosomes is in most cases obtained by the addition of the phospholipid DOPE, which as a result of its structure can induce inverted hexagonal liquid crystalline phases (J. O. Rädler et al., Science 281, 78-81 (1998)). These exhibit a high tendency to fuse with bilayer structures (e.g. biological membranes). Because of the reasons stated above it is of crucial importance to prevent the acidification in the endosomes in order to retard the fusion between endosomes and lysosomes. This fact is supported by the observation, that the addition of 10-100 μM chloroquine—a weak base—to the cell culture medium significantly increases the efficiency of the cationic amphiphiles (P. L. Felgner et al., J. Biol. Chem. 269 (4), 2550-2561 (1994); A. K. Tanswell et al., Am. J. Physiol. 275 (3 Pt 1) L452-L460 (1998)). The addition of a weak base with a buffering capacity also reduces the activity of the degrading enzymes in the lysosomes which show optimal activity in an acidic pH range. However, this approach cannot be applied in vivo.

Since the first publication by Felgner a great number of cationic amphiphiles have been synthesized for the transfer of anionic macromolecules (as e.g. DNA) (A. D. Miller, Angew. Chem. 110, 1862-1880 (1998); L. Huang, X. Gao, Gene Therapy 2, 710-722 (1995)). Most cationic amphiphiles were found empirically. The disadvantage of many of these cationic lipids as e.g. DOTMA or DLRIE is the fact that they are difficult to metabolize and therefore show toxic properties in cells. Furthermore, except for derivatives of lipopolyamine (Lipospermine and others) (Blagbrough et al., Chem. Commun. 13, 1403-1404 (1998) the presently known cationic amphiphiles do not exhibit sufficient buffering capacity at physiological pH (approximately pH 7.4) to prevent acidification of the endosomes. The lipopolyamine compounds are multifunctional molecules with only slight differences in the chemical reactivity of these functions. Therefore, the synthesis of these molecules requires orthogonal strategies to protect the functional groups in the different synthesis steps making their production difficult. Another important drawback of the presently known cationic lipids is the fact that their transfection activity is inhibited already in presence of small amounts of serum (>5%) in the medium.

The challenge for this invention was to design a new amphiphilic polyamine for the transfer of biopolymers (in particular DNA and RNA) into eucaryotic cells with the following properties:

Easy to metabolize and low toxicity to cells

High transfer efficiency also in presence of high serum content in the medium

Easy and inexpensive to produce in high quantities

Molecule with combined functionalities that permits efficient complexing of anionic macromolecules and high buffering capacity at physiological pH

A surprisingly efficient transfer of biomolecules (in particular DNA and RNA) into eucaryotic cells (transfection) with the above mentioned properties could be achieved with the invented polyamine of the general chemical structure given in (I)

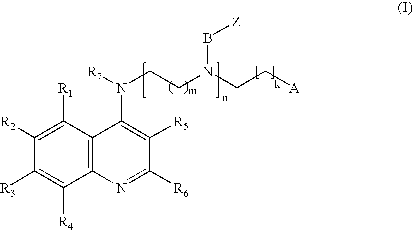

wherein R₁ to R₆ can be independent of each other: H, halogen, —C≡N, —NO₂, —SO₃H, —COOH, —N(alkyl)₂, —NH(alkyl), —NH₂, -alkyl, —OH, —O-alkyl, —O-aryl, —O-hetaryl, —O(C═O)alkyl, (C═O)alkyl, —SH, —S-alkyl,

R₇ is either H or an alkyl group with 1 to 4 carbon atoms

and m and k are independent of each other an integer from 1 to 6,

n is an integer from 1 to 3,

A is a group N⁺R₈R₉R₁₀Y⁻, wherein R₈ to R₁₀ are independent of each other either H, an alkyl group with 1 to 4 C-atoms, a group —(CH₂)ᵢ—OH or a group —(CH₂)ᵢ—NH₂ with i=2 to 6 and Y⁻ being a pharmaceutically acceptable anion,

B is a group

—CH₂— or

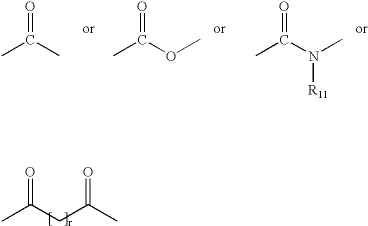

wherein R₁₁ has the same meaning as R₇ and r represents an integer from 1 to 6,

Z denotes a steroid bound via C-atom 3 (of the sterane backbone), a group R₁₂ or a group

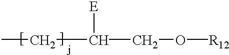

with j being an integer from 0 to 4, R₁₂ being a saturated or unsaturated alkyl or acyl group with 8 to 24 C-atoms and E being a group O—R₁₃ or CH₂—O—R₁₃ wherein R₁₃ has the same meaning as R₁₂ and can be the same as or different to R₁₂,

and wherein the nitrogen atom of the quinoline ring can be protonated by an acid H⁺Y⁻ with Y⁻ being a pharmaceutically acceptable anion.

Preferred are compounds with R₁, R₂, R₄, R₆ and R₇ being a hydrogen atom, R₅ a hydrogen or alkyl group and R₃ a halogen atom, particularly Cl. Furthermore, preferred are compounds in which m and k are equal 1 to 3, n equals one and A is either a dimethylammonium- or diethylammonium group. Further are compounds preferred where B is either

—CH₂— or

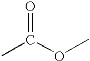

and Z represents a membrane associated steroid or a 1,2-diglyceride like structure.

Most preferred are molecules where Z denotes a cholesteryl or a 1,2-dioleoyloxyethyl group and the pharmaceutically acceptable anion Y⁻ is halogen, acetate, or phosphate.

In a preferred application the invented lipid is mixed with other lipids known to experts in the field, such as phospholipids in particular DOPE or membrane associated steroids, in particular cholesterol and employed to shuttle biologically active biomolecules, such as DNA and RNA into cells. The lipids can be in an aqueous (liposomal) suspension or dissolved in solvents miscible with water and the DNA (RNA) can be pre-complexed with protamine sulfate.

As compared to the known transfection reagents the invented compounds exhibit a number of advantages. Molecules as described in this invention possess at least two basic functions with clearly different pKₛ in the hydrophilic head group. One in the aromatic ring (quinoline ring) the other the not lipophilic modified nitrogen in the aliphatic chain. The pKₛ values are approximately pKₛ=10.2 for the diethylamino group and pKₛ=8.06 for the quinoline. At the physiological pH of 7.4 the nitrogen of the diethylamino group is nearly fully protonated and can provide a strong electrostatic interaction with the negatively charged biomolecule. The positive charge in compounds with a quarternary amine is virtually independent of the pH-value of the medium. In contrast, the nitrogen in the quinoline base at pH 7.4 is approximately 20% deprotonated and therefore capable to provide buffer capacity following uptake into the endosomes, thereby preventing acidification of the endosomes. An additional advantage is the fact that some quinoline-bases can inhibit H⁺-ATPases. Therefore, the compounds presented in this invention could also inhibit the H⁺-ATPase by the quinoline-base mechanism, thereby preventing acidification in the endosomes and inhibiting transport of the aggregates to the lysosomes which would result in their subsequent digestion. In addition the aromatic character of the quinoline structure permits interactions with the π-electrons of the nucleotid bases of the DNA and RNA providing possibly an additional advantage for the complex formation as compared to other known reagents.

The transfection reagents used so far do not contain a chromophore and are therefore difficult to detect during the purification and in analytical steps that are based on HPLC-methods. The strong UV absorption of the quinoline backbone at 200-300 nm makes detection of the present invented reagent easy, providing a considerable advantage for the analytical work and for the preparation as pharmaceuticals.

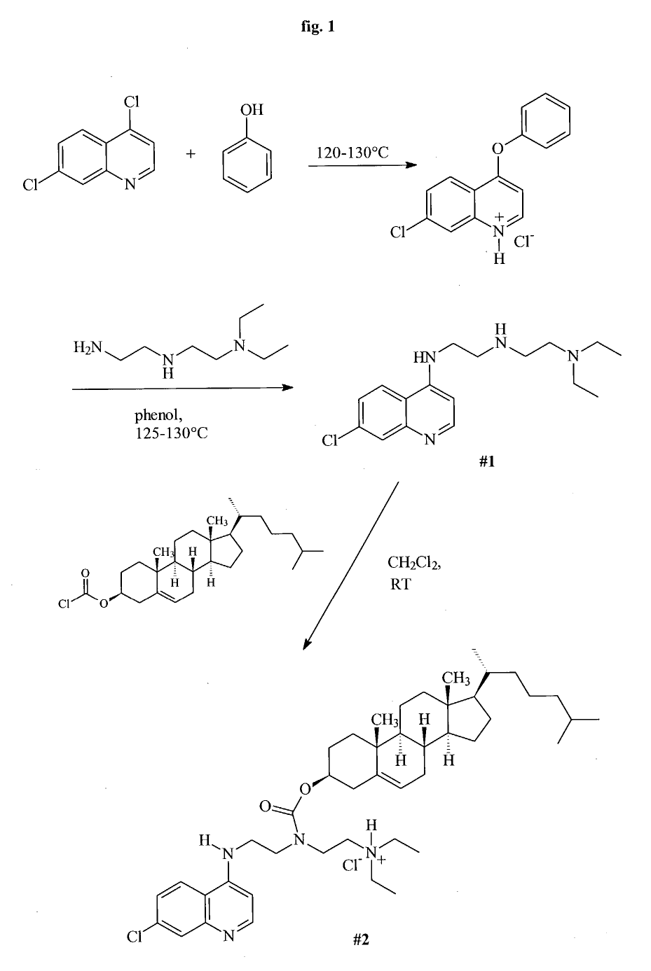

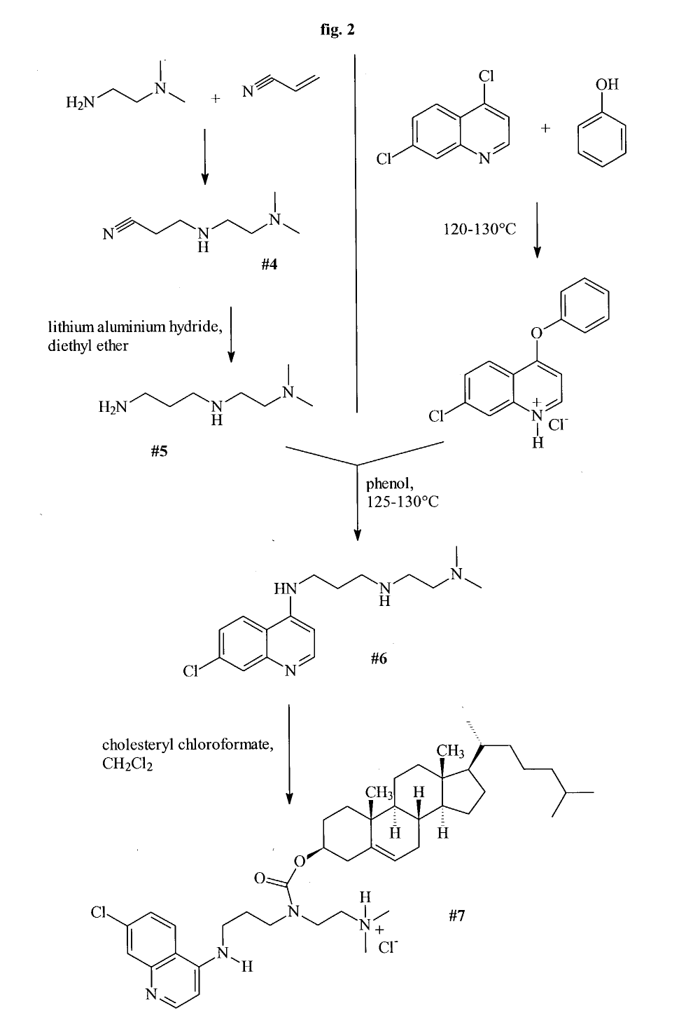

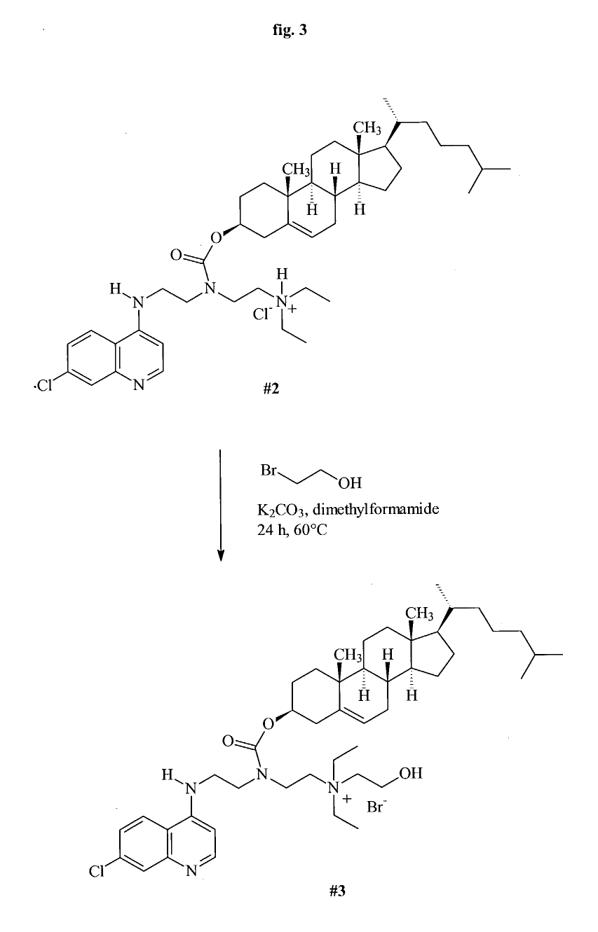

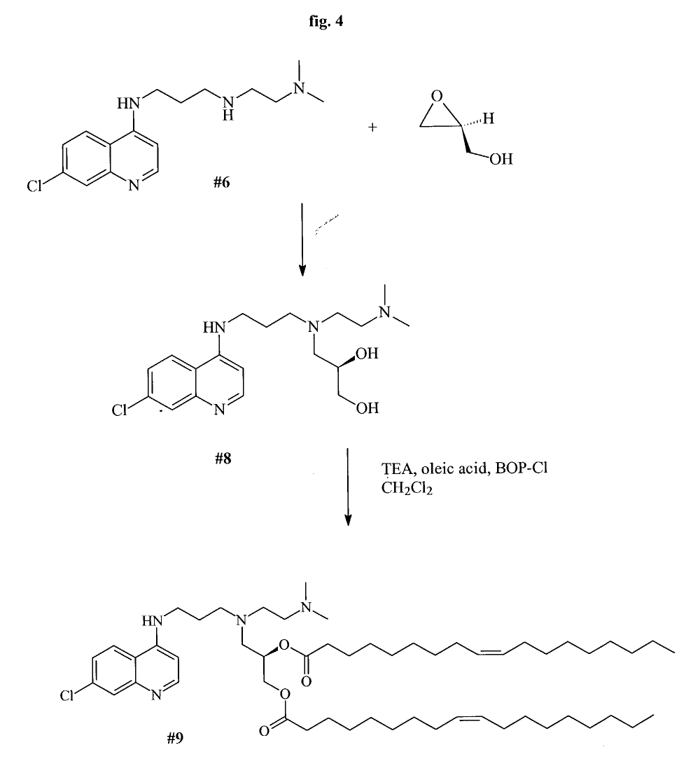

The synthesis of the invented compound requires only few steps and is using inexpensive starting material and known synthesis methods for the expert in the field (Methoden der organischen Chemie (Houben-Weyl) 4. Aufl. Thieme Verlag (Stuttgart) 1952; The Chemistry of Heterocyclic Compounds, John-Wiley & Sons, Inc, Vol. 32 part 1 (1977)). The relevant reaction schemes are shown in FIGS. 1-4.

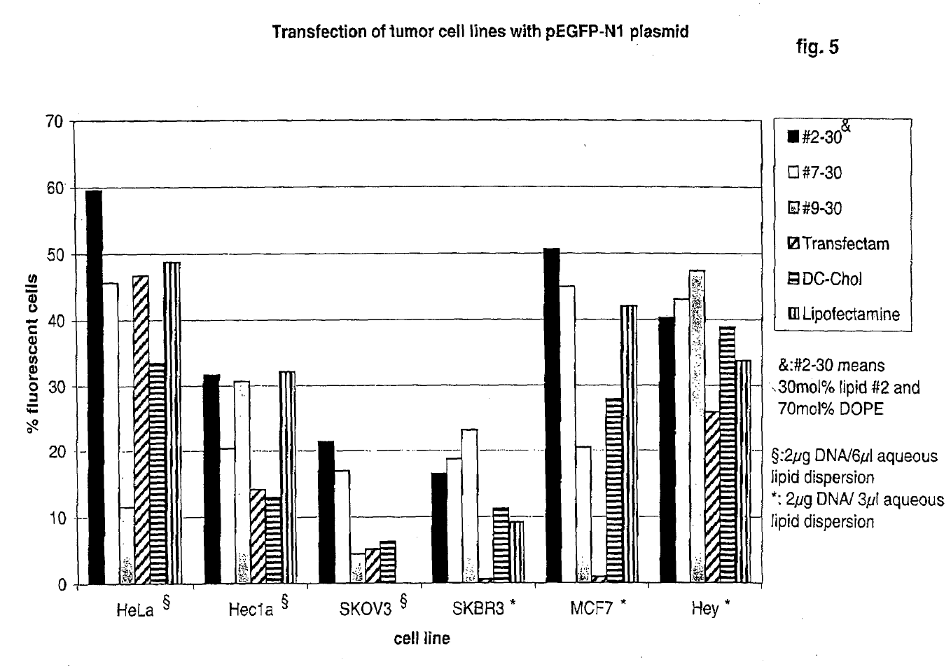

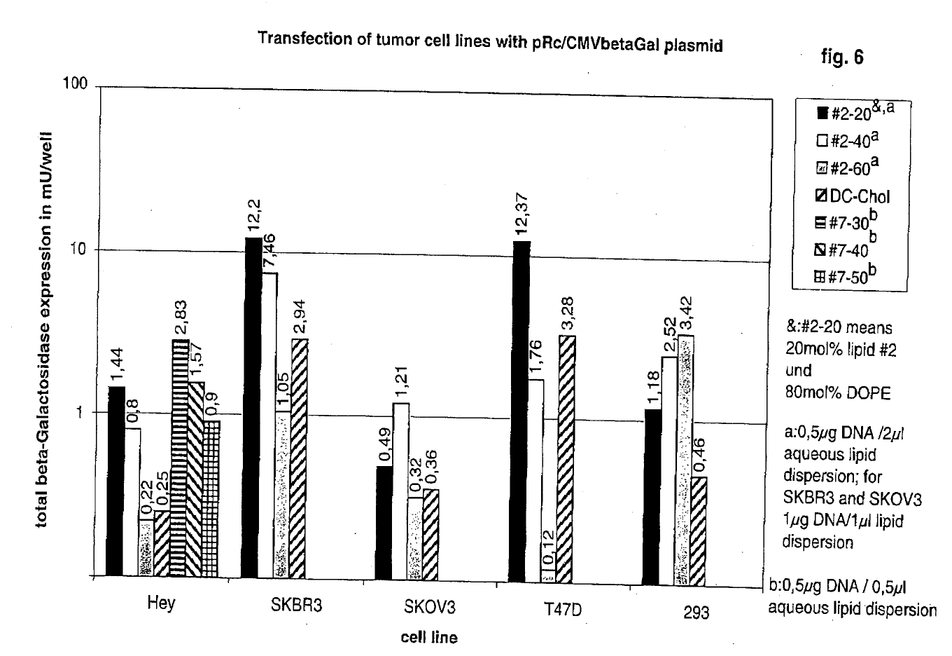

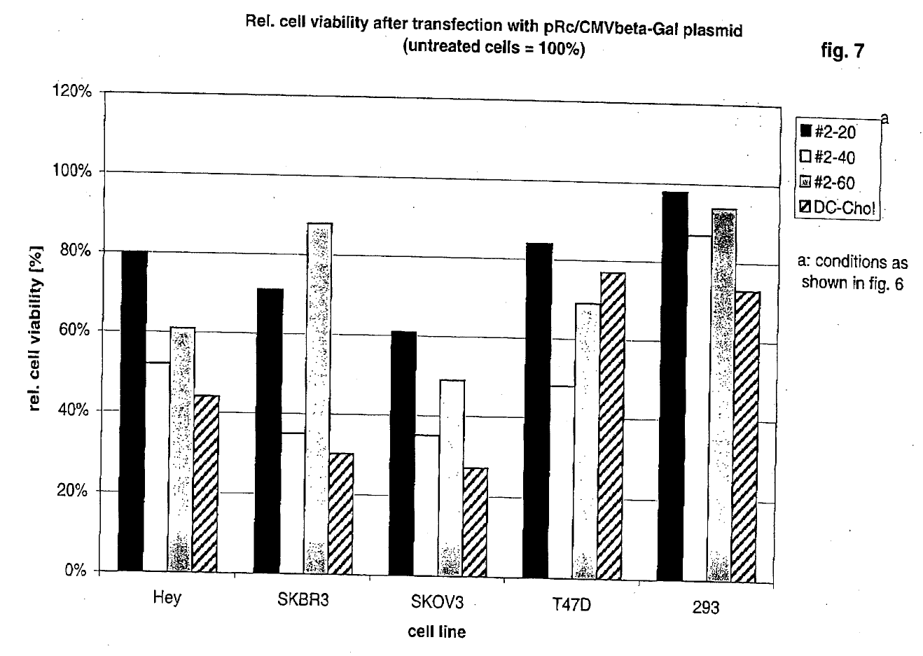

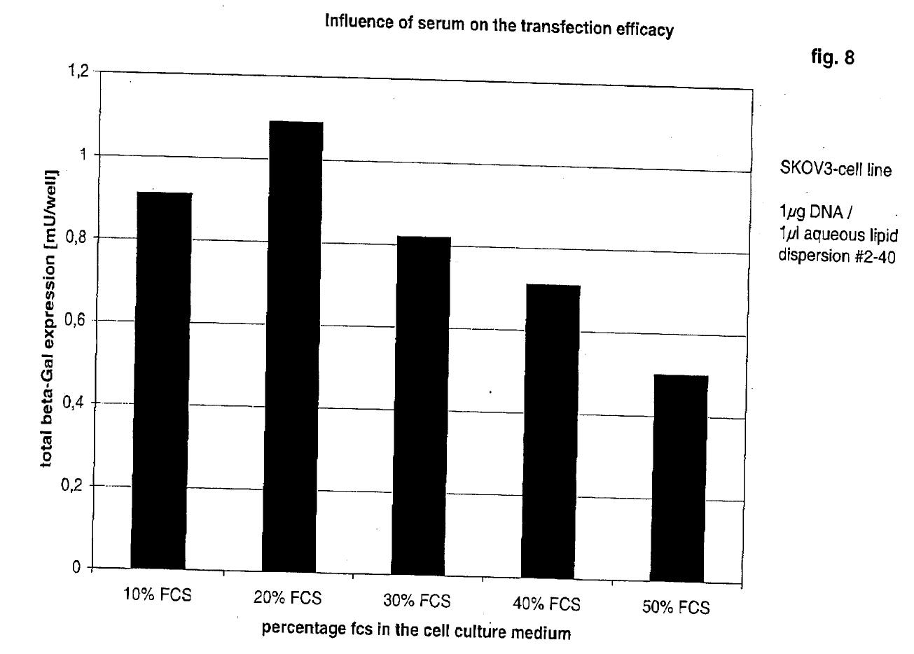

The transfection potency and the toxicity of the invented lipid and lipid mixtures were evaluated in different tumor cell lines and compared to known transfection reagents (FIGS. 5 and 6). GFP and β-galactosidase plasmids were used as reporter genes. The lipids #2, #7, #9 were mixed with the phospholipid 1,2-dioleoyl-sn-glycero-3-phosphoethanolamine (DOPE). Aqueous liposomal formulations as well as ethanolic solutions proved to be equally suitable for transfection. Using the lipids #2, #7 and #9 it was possible to transfect all tested cell lines with a higher efficiency than with the known lipid formulations Transfectam®, Lipofectamine™ and DC-Chol. The low toxicity profile (FIG. 7) and the lack of transfection inhibition by serum in the medium were most surprising and advantageous (FIG. 8). Even in presence of 50% fetal calf serum (FCS) the gene transfer efficiency was still higher than 50% which is normally observed under the standard conditions with only 10% FCS.

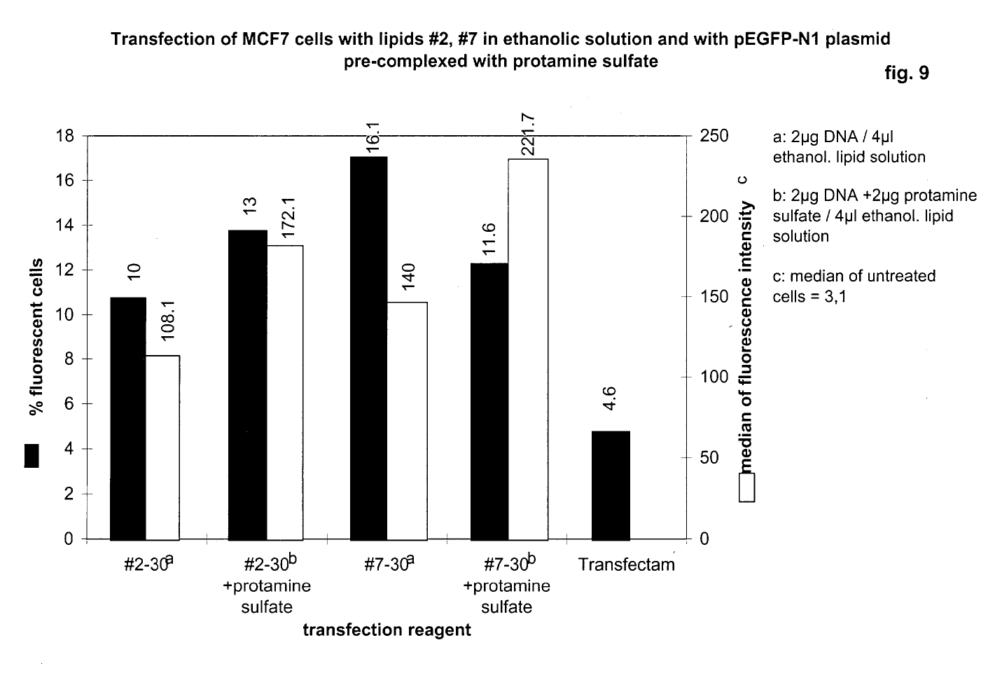

The transfection efficiency of the lipids #2 and #7 (mixtures with 70 mol % DOPE) in ethanolic solutions are presented in FIG. 9. With this mixtures for example the breast tumor cells MCF7 can be transfected significantly more efficient with pEGFP-N1-plasmid than with the known reagent Transfectam. The pre-complexing of the DNA with the poly-cationic protein protamine sulfate is leading to a significant increase in the percentage of transfected cells. Surprisingly this was leading to a considerably stronger expression of the reporter gene. The median of fluorescence intensity of the transfected cells following pre-complexing of the DNA with protamine sulfate was more than 50% higher than obtained with DNA without pre-complexing (FIG. 9).

The invention is further described in the following examples. These examples represent preferred compositions, however the scope of the invention is not restricted to these forms in any way.

### EXAMPLE 1

Synthesis of 7-chloro-4-[N¹-(N⁴-H)-(N⁷-diethyl)-1,4,7-triaza-heptyl]-quinoline (#1)

In a 25 ml round bottom flask 1.39 g (7 mmol) 4,7-dichloroquinoline and 1.32 g (14 mmol) phenol were stirred for one hour at 120° C. under an argon atmosphere. When the melt was cooled down to 60° C. 1.115 g (7 mmol) diethylaminoethyl-ethylenediamine was added and the mixture was stirred for 24 hours at 125° C. During this time it turned dark red/brown. After cooling to room temperature the residue was dissolved in 30 ml 2N acetic acid and was acidified to pH 4-5 with conc. acetic acid. The solution was extracted 4 times with 25 ml of CHCl₃ each and the pH of the aqueous layer was adjusted to pH 8-9 with conc. ammonia (7-8 ml). The thus obtained cloudy solution was extracted 4× with 25 ml of CHCl₃ each. The combined organic layers were washed with 10 ml of saturated NaHCO₃-solution, dried over Na₂SO₄ and the solvent was removed on a rotary evaporator. After drying in vacuo 1.94 g (6.1 mmol)=87% of a brown viscous oil was obtained which slowly solidified. The thus obtained compound is suitable for the preparation of compound #2 without further purification. Purification by preparative column chromatography on silica (70-230 mesh) with a gradient comprising of CH₂Cl₂/MeOH 3:1 (0.1% triethylamine) to CH₂Cl₂/MeOH 1:3 (0.1% triethylamine) afforded the analytically pure substance.

TLC (silica 60) [CHCl₃/MeOH 4:1]; R_{f}=0.04; UV-detection

### EXAMPLE 2

Synthesis of 7-chloro-4-[N¹-(N⁴-carboxycholesteryl)-(N⁷-diethyl)-1,4,7-triaza-heptyl]-quinoline Hydrochloride (#2)

In a 50 ml round bottom flask 614 mg (1.91 mmol) of #1 was dissolved in 15 ml dry dichloromethane under an argon atmosphere. After that a solution of 853 mg (1.90 mmol) cholesterylchloroformate in 20 ml of dry dichloromethane was added dropwise during 15 min at room temperature. After stirring for 2 h the solvent was removed on a rotary evaporator and the residue was dried in vacuo for further two hours yielding 1.45 g of a yellowish amorphous solid. Subsequent column chromatography on silica (70-230 mesh) with CH₂Cl₂/MeOH 7:1 as the eluent afforded the pure compound as a colorless amorphous solid.

TLC (silica 60) [CH₂Cl₂/MeOH 4:1]; R_{f}=0.24; UV-detection and detection with Vanillin/conc. sulfuric acid

### EXAMPLE 3

Synthesis of 7-chloro-4-[N¹-(N⁴-carboxycholesteryl)-(N⁷-diethyl-hydroxyethyl)-1,4,7-triaza-heptyl]-quinoline hydrobromide (#3)

In a 10 ml round bottom flask 40 mg (52 μmol) of #2 was dissolved in 500 μl dry dimethylformamide under an argon atmosphere and 30 mg dry sodium carbonate and 300 μl 2-bromoethanol were added. After stirring the mixture for 16 h at 65° C. 10 ml dichloromethane were added and the solids were removed by filtration. After that the solvents were removed in vacuo at 60° C. and the residue was further purified by column chromatography on silica (70-230 mesh) with dichloromethane/methanol 5:1 as eluent yielding 25 mg (29.1 μmol)=56% of a colorless glassy solid

TLC (silica 60) [CH₂Cl₂/MeOH 3:1]; R_{f}=0.25; UV-detection and detection with Vanillin/conc. sulfuric acid

### EXAMPLE 4

Synthesis of N,N-dimethyl-N′-cyanoethyl-ethylenediamine (#4)

In a 500 ml round bottom flask 10.9 ml (100 mmol) 2-dimethylaminoethylamine were dissolved in 150 ml 2-propanol. The solution was chilled in an ice bath and 6.91 ml (105 mmol) acrylonitrile were added. After that the mixture was stirred for 72 h at room temperature. The solvent was removed in vacuo (7 mbar) at 40-50° C. and the residue was then distilled in vacuo yielding 9.80 g (69 mmol)=69% of a colorless liquid with a bp. of 135° C. (7 mbar).

IR (film, [cm⁻¹]): \tilde{ν}=3500-3150 (m, b) [ν N—H]; 2930 (s), 2840 (s), 2810 (s), 2750 (s) [ν_{as,s} CH₂, CH₃]; 2240 (s) [ν CN]; 1460 (m), 1440 (m, sh) [δ_{as,s} CH₂, δₐₛ CH₃]

¹H-NMR (400.132 MHz, CDCl₃ [ppm]): δ=1.35-1.6 (s(b), 1H, NH); 2.13 (s, 6H, N(CH₃)₂); 2.17, 2.42, 2.62, 2.85 (4×t, 8H, 4×CH₂)

¹³C{¹H}-NMR (100.625 MHz, CDCl₃ [ppm]): δ=18.5, 45.1, 46.4, 58.7 (4×CH₂); 45.25 (N(CH₃)2); 118.5 (CN)

### EXAMPLE 5

Synthesis of N⁸-dimethyl-1,5,8-triazaoctane (#5)

In a 250 ml three-necked flask with a magnetic stirrer, condenser and septum 90 ml of a 1M solution of lithium aluminium hydride in diethyl ether were introduced under argon. After that 9.93 g (70 mmol) #4 were added dropwise via a syringe so that the ether is only slightly boiling. After refluxing the mixture for 5 hours 20 ml of a 20% sodium hydroxide solution were added very carefully.

The thus precipitating hydroxides can be easily removed by filtation. The hydroxides were extracted five times with each 40 ml of boiling diethyl ether whereupon the solvent is removed on a rotary evaporator. The residue was dissolved in 150 ml dichloromethane and was washed once with 5 ml of 5N NaOH-solution. After drying with sodium sulfate the solvent was removed on a rotary evaporator and the thus obtained oily liquid was fractionally distilled in vacuo yielding 3.96 g (27.3 mmol)=39% of a colorless liquid with a bp. of 65° C. (0.1 mbar).

IR (film, [cm⁻¹]): \tilde{ν}=3600-3100 (s, b) [ν N—H]; 2920 (s), 2800 (s), 2750 (s) [V_{as,s} CH₂, CH₃]; 1445 (s) [δ_{as,s} CH₂, δₐₛ CH₃]

### EXAMPLE 6

Synthesis of 7-chloro-4-[N¹-(N⁵-H)-(N⁸-dimethyl)-1,5,8-triaza-octyl]-quinoline (#6)

In a 25 ml round bottom flask 812 mg (4.1 mmol) 4,7-dichloroquinoline and 1.16 g (12.3 mmol) phenol were stirred for one hour at 125-130° C. under an argon atmosphere. Following cooling of the mixture to 60° C. 596 mg (4.1 mmol) N⁸-dimethyl-1,5,8-triazaoctan (#5) was added and the mixture was stirred for 24 hours at 130° C. Thereupon the mixture was cooled to room temperature and was dissolved in 20 ml 5N sodium hydroxide. The solution was transferred to a separatory funnel and was extracted two times with 60 ml of dichloromethane each. The combined organic layers were washed with 10 ml 5N sodium hydroxide, dried over sodium sulfate and the solvent was removed on a rotary evaporator yielding 1.37 g of a yellowish viscous oil. The thus obtained compound is suitable for the preparation of compound #7 without further purification. Purification by preparative column chromatography on silica (70-230 mesh) with a gradient comprising of CH₂Cl₂/MeOH 3:1 (0.1% triethylamine) to CH₂Cl₂/MeOH 1:3 (0.1% triethylamine) yielded the analytically pure substance.

TLC (silica 60)

[CH₂Cl₂/MeOH 3:1 (0.1% triethyl amine)]; R_{f}=0.07; UV-detection

[80% ethanol (2% triethyl amine)]; R_{f}=0.11; UV-detection

### EXAMPLE 7

Synthesis of 7-chloro-4-[N¹-(N⁵-carboxycholesteryl)-(N⁸-dimethyl)-1,5,8-triaza-octyl]-quinoline Hydrochloride (#7)

In a 50 ml nitrogen flask 580 mg (1.89 mmol) of #6 were dissolved in 40 ml dry dichlormethane under an argon atmosphere. After chilling the solution in ice 850 mg (1.89 mmol) cholesteryl chloroformate were added. The mixture was stirred at room temperature for 3 hours whereupon the solvent was removed on a rotary evaporator. Subsequent purification by column chromatography on silica 60 (70-230 mesh) with an CH₂Cl₂/MeOH 7:1 eluent yielded the pure compound as a colorless, amorphous solid.

TLC (silica 60) [CH₂Cl₂/MeOH 7:1]; R_{f}=0.19; UV-detection and detection with Vanillin/conc. sulfuric acid

### EXAMPLE 8

Synthesis of 7-chloro-4-[N¹-(N⁵-(2(R),3-dihydroxy)propyl)-(N⁸-dimethyl)-1,5,8-triaza-octyl]-quinoline (#8)

In a stoppered 10 ml round bottom flask 244 mg (795 μmol) of #6 were dissolved in 2 ml dry methanol. The solution was stirred at 0-4° C. for 72 hours and during this time 100 μl (=1.51 mmol) (S)-glycidol were added in portions of 10 μl. After that the volatile components were removed on a rotary evaporator and the residue was purified by column chromatography on 20 g silica 60 (70-230 mesh) using a gradient comprising of CH₂Cl₂/MeOH 3:1 (0.1% triethyl amine) to CH₂Cl₂/MeOH 1:1 (0.1% triethyl amine). The product was obtained in a yield of 24.5 mg (64 μmol)=8.1% as a colorless very viscous oil.

TLC (silica 60) [80% ethanol (2% triethyl amine)]; R_{f}=0.27; UV-detection

### EXAMPLE 9

Synthesis of 7-chloro-4-[N¹-(N⁵-(2(R),3-dioleoyloxy)propyl)-(N⁸-dimethyl)-1,5,8-triaza-octyl]-quinoline (#9)

In a stoppered 10 ml round bottom flask 24.5 mg (64 μmol) of #8 were dissolved in 1.5 ml dry dichloromethane and 36 μl (256 μmol) triethyl amine, 41 μl (128 μmol) oleic acid and 34 mg (131 μmol) N,N-bis-[2-oxo-3-oxazolidinyl]-phosphoric acid-diamide chloride (BOP-Cl) were added. The mixture was stirred at room temperature for 48 hours and the progress of the reaction was followed by TLC (dichloromethane/methanol 7:1). After completion of the reaction the solvent was removed on a rotary evaporator and the residue was purified by column chromatography on 20 g of silica 60 (70-230 mesh) with an dichloromethane/methanol 7:1 eluent. The product was obtained in a yield of 24 mg (26 μmol)=41% as a colorless waxy solid. TLC (silica 60) [dichloromethane/methanol 7:1]; R_{f}=0.30; UV-detection and detection with Vanillin/conc. sulfuric acid

### EXAMPLE 10

Formulation of the Amphiphilic Polyamines for the Transfection Experiments According to the Invention.

a) Liposomal Formulation

Solutions of the amphiphiles #2, #3, #7, #9 in chloroform were mixed with a solution of 1,2-dioleoyl-sn-glycero-3-phosphoethanolamine (DOPE) in chloroform in different molar ratios and were dried in vacuo to obtain a lipid film. Residual solvent was removed in high vacuo. Thereupon the lipid films were rehydrated in sterile water and liposomes were generated by sonification. The total lipid concentration of the thus obtained dispersion was 1 mg/ml.

b) Ethanolic Formulation

The cationic lipids were mixed in different molar ratios with DOPE and were dissolved in dry ethanol so that the total lipid concentration of the resulting solution was 1 mg/ml.

EXAMPLE 11

Transfection of Adherent Cell Lines

In general: The cell lines used HeLa, Hec1a, SKOV3, MCF-7, Hey, SKBR3, T47D, 293 were cultivated according to standard ATCC conditions. Commercial transfection reagents Transfectam®, Lipofectamine™ und Superfect were used according to the manufacturer's instructions. DC-Chol was synthesized as described previously [L. Huang et al. Biochem. Biophys. Res. Commun. 179(1) (1991) 280-285] and was formulated with DOPE for the transfection experiments.

a) Transfection with pEGFP-N1-Plasmid

a. 1) Usage of the Aqueous Liposomal Dispersions

The day before the transfection 40-50000 cells were seeded per well in a 24-well cell culture plate (appr. 70% confluence). For the preparation of the complexes 3 μl (for SKBR3, MCF-7, Hey) respectively 6 μl (for HeLa, Hec1a, SKOV3) of the aqueous lipid reagent (30 mol % lipid #2 respectively #7 respectively #9; 70 mol % DOPE) were added to a solution of each 2 μg of the plasmid-DNA (pEGFP-N1; Clontech Laboratories, Inc; Katalog-Nr. 6085-1) in 300 μl of fcs-free medium and were incubated at room temperature for 30 min. Shortly before the transfection the cell culture medium was replaced with 700 μl fresh medium (14.3% fcs). After addition of the lipid-DNA complexes and incubation of the cells for 48 hours the percentage of GFP expressing fluorescent cells was determined by FACS-analysis. For this the cells were washed with PBS and were separated by trypsination and subsequent centrifugation. The thus obtained cell pellet was resuspended in 500 μl PBS and the percentage of fluorescent cells was determined by FACS-analysis using a FACScan device (Becton Dickinson).

a.2) Usage of the Ethanolic Lipd Solutions

The day before the transfection 40-50000 cells were seeded per well in a 24-well cell culture plate (appr. 70% confluence). For the preparation of the complexes 4 μl of the ethanolic lipid reagent (30 mol % lipid #2 respectively #7; 70 mol % DOPE) were added to a solution of each 2 μg of the plasmid-DNA (pEGFP-N1; Clontech Laboratories, Inc; catalog-nr. 6085-1) in 300 μl fcs-free medium and were incubated at room temperature for 30 min. For the transfection in the presence of protamine sulfate 2 μg plasmid-DNA was pre-complexed with 2 μg protamine sulfate in 300 μl serum free medium for 10 min whereupon 4 μl of the lipid reagent were added. Shortly before the transfection the cell culture medium was replaced with 700 μl fresh medium (14.3% fcs). After addition of the lipid-DNA complexes and incubation of the cells for 48 hours the percentage of GFP expressing fluorescent cells was determined by FACS-analysis as described above.

b) Transfection with pRc/CMVlacZ-Plasmid pRc/CMVlacZ-plasmid: the lacZ gene was cut from the commercially available vector pSVbetaGal (Promega; cat.-nr. E1081) using the restriction enzymes HindIII and XbaI and was cloned into the expression plasmid pRc/CMV (Invitrogen; cut with HindIII and XbaI).

The day before the transfection 12-15000 cells were seeded per well in a 96-well cell culture plate. Shortly before the transfection the medium was replaced with 80 μl/well of fresh medium (20% fcs), whereas for the examination of the influence of fcs on the transfection efficiency (FIG. 8) medium with 20-100% fcs was employed.

Thereupon 80 μl of the lipid-DNA complex, which was prepared by mixing a solution of 0,5-1%g DNA in 40 μl fcs free medium with a solution of 0.5-2 μl of the aqueous lipid dispersion (20-60 mol % lipid #2 or #7; 80-40 mol % DOPE) in 40 μl fcs free medium, were added and the cells were incubated for 48 h. DC-Chol was used as described in the literature. The simultaneous determination of the reporter gene expression and the cell viability was carried out according to D. Groth, O. Keil et al.; Anal. Biochem. 258 (1998) 141-143. This method afforded the total beta-Galactosidase expression in mU/well and the cell viability in relation to untreated cells.

1. A compound of the general chemical structure 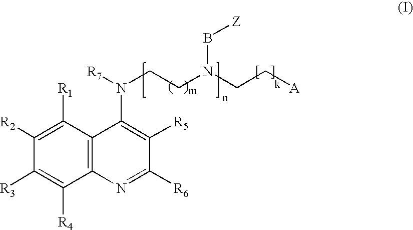
wherein R₁ to R₆ can be independent of each other: H, halogen, —C≡N, —NO₂, —SO₃H, —COOH, —N(alkyl)₂, —NH(alkyl), —NH₂, —alkyl, —OH, —O-alkyl, —O-aryl, —O(C═O)alkyl, (C═O)alkyl, —SH, —S-alkyl, and wherein alkyl represents an aliphatic group with 1 to 4 carbon atoms and aryl represents a phenyl or benzyl moiety,
R₇ is either H or an alkyl group with 1 to 4 carbon atoms
and m and k are independent of each other an integer from 1 to 6,
n is an integer from 1 to 3,
A is a group N⁺R₈R₉R₁₀Y⁻, wherein R₈ to R₁₀ are independent of each other either H, an alkyl group with 1 to 4 C-atoms, a group —(CH₂)ᵢ—OH or a group
—(CH₂)ᵢ—NH₂ with i=2 to 6 and Y⁻ being a pharmaceutically acceptable anion,
B is a group
—CH₂— or 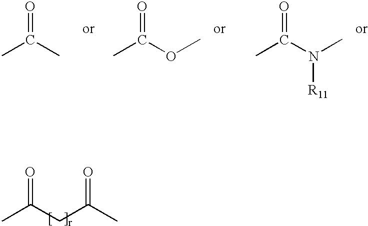
wherein R₁₁ has the same meaning as R₇ and r represents an integer from 1 to 6,
Z is a steroid bound via C-atom 3 (of the sterane backbone), a group R₁₂ or a group 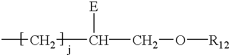
with j being an integer from 0 to 4, R₁₂ being a saturated or unsaturated alkyl or acyl group with 8 to 24 C-atoms and E being a group O—R₁₃ or CH₂—O—R₁₃ where R₁₃ has the same meanig as R₁₂ and can be the same as or different to R₁₂,
and where the nitrogen atom of the quinoline ring can be protonated by an acid H⁺Y⁻ with Y⁻ being a pharmaceutically acceptable anion.
with j being an integer from 0 to 4, R₁₂ being a saturated or unsaturated alkyl or acyl group with 8 to 24 C-atoms and E being a group O—R₁₃ or CH₂—O—R₁₃ where R₁₃ has the same meanig as R₁₂ and can be the same as or different to R₁₂,
and where the nitrogen atom of the quinoline ring can be protonated by an acid H⁺Y⁻ with Y⁻ being a pharmaceutically acceptable anion.

2. Compounds according to claim 1 wherein R₁, R₂, R₄, R₆ and R₇ represents a hydrogen atom, R₅ represents a hydrogen or alkyl group and R₃ represents a halogen atom.

3. Compounds according to claim 1 or 2 wherein m and k are equal 1 to 3, n equals one and A is either a dimethylammonium- or diethylammonium group.

4. Compounds according to claim 1,2 or 3 wherein B represents a group being either —CH₂— or —C(O)O— and
Z represents a cholesteryl- or a 1,2-dioleoyloxylethyl-moiety.

5. Compounds according to claim 1 to 4 wherein Y⁻ is either a halogen, acetate or phosphate anion.

6. Pharmaceutical or diagnostic application of a reagent for the transfer of biologically active, anionic macromolecules into eucaryotic cells, where the reagent is containing at least one compound listed in claims 1 to 5 and forms aggregates or particles with biologically active anionic macromolecules and is brought in contact with cells in vitro or in vivo. Additional lipidic compounds may be added to different amounts.

7. Pharmaceutical or diagnostic application of a reagent for the transfer of biologically active anionic macromolecules in eucaryotic cells as in claim 6 where the biologically active anionic molecule is DNA, RNA, antisense-DNA, antisense-RNA, ribozyme, peptides or proteins.

8. Pharmaceutical or diagnostic application of a reagent for the transfer of biologically active anionic macromolecules in eucaryotic cells as in claim 6 or 7 wherein the lipidic compounds added are in the class of phospholipids or steroids and in which 1,2-diolcoyl-sn-glycero-3-phosphoethanolamine is particularly useful.

9. Pharmaceutical or diagnostic application of a reagent for the transfer of biologically active anionic macromolecules in eucaryotic cells as in claim 6 to 8 where the reagent is a dispersion in aqueous medium or in solution in a solvent miscible with water and where, in the case of an aqueous suspension, a cryo-protectant from the group of lactose, trehalose, sucrose, glucose, fructose, galactose, maltose, mannitol or polyethyleneglycol may also be dissolved in the mixture.

10. Pharmaceutical or diagnostic application of a reagent for the transfer of biologically active anionic macromolecules in eucaryotic cells as in claim 6 to 9 where the biologically active anionic molecule is pre-complexed with polycationic molecules of the class of spermine, spermidine, protamine sulfate, histone H1, histone H2A, histone H2B, histone H3, histone H4, HMG1 or HMG17 protein.
